SEQUENCE LISTING (3 sequences)

SEQ ID NO: 1 - DNA, 1518 bp, Homo sapiens
  CDS (41)..(661)
aaaacagcccggagcctgcagcccagccccacccagacccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgtgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctcca

SEQ ID NO: 2 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - protein, 177 aa, Homo sapiens
TPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2012/0121535 A1
(43) Pub. Date: May 17, 2012

(54) AGENT FOR PREVENTING RECURRENCE OF LEUKEMIA

(75) Inventors: Fumihiko Ishikawa, Kanagawa (JP); Yoriko Saito, Kanagawa (JP); Leonard D. Shultz, Bar Harbor, ME (US)

(73) Assignees: RIKEN, Wako-shi (JP); Leonard D. SHULTZ, Bar Harbor, ME (US)

(21) Appl. No.: 13/254,537

(22) PCT Filed: Mar. 5, 2010
(86) PCT No.: PCT/JP2010/053685

§ 371 Date: Jan. 18, 2012

(30) Foreign Application Priority Data
Mar. 5, 2009 (JP) 2009-052723

(51) Int. Cl. A61K 38/19; A61P 35/02

(52) U.S. Cl. 424/85.1

(57) ABSTRACT

The present invention provides a drug capable of initiating the progression of the cell cycle of leukemia stem cells to overcome the resistance of the leukemia stem cells to cell cycle-dependent chemotherapeutic agents, and a drug for suppressing recurrence of leukemia containing the same, and the like, an agent containing G-CSF, wherein the agent is for inducing the progression of the cell cycle of leukemia stem cells, a drug for suppressing recurrence of leukemia containing a combination of G-CSF and a cell cycle-dependent antitumor agent, and the like.

### TECHNICAL FIELD

The present invention relates to a drug capable of initiating the progression of the cell cycle of leukemia stem cells to overcome the resistance of the leukemia stem cells to cell cycle-dependent chemotherapeutic agents, and an agent for suppressing recurrence of leukemia comprising the same, and the like.

### BACKGROUND ART

Acute myelogenous leukemia (AML) is the most highly frequent (onset rate) adult leukemia, characterized by the clonal expansion of immature myeloblasts initiating from rare leukemic stem cells (LSCs) (non-patent documents 1-3).

Conventional chemotherapeutic agents have been posing the difficult problem of being unable to rescue patients from AML because of its recurrence after temporary remission. Therefore, to develop an effective therapeutic agent and therapeutic method, there has been a strong demand for elucidating the mechanism of recurrence by clarifying the properties of leukemia, including the functional features and molecular features of LSCs.

The present inventors have created a novel immunodeficient strain with improved long-term xenogeneic engraftment, NOD.Cg-Prkdc^{scid}Il2rg^{tm1Wjl/J} (NOD/SCID/IL2rg^{null}) mice, carrying a complete null mutation (non-patent document 4) of the common γ chain (non-patent document 5). This strain has life expectancy of >90 weeks, and has been clarified to be able to more accurately assess the engraftment and lymphoid/myeloid differentiation capacity of human long-term repopulating HSCs (LT-HSCs) than strains such as NOD/SCID (non-patent document 6), NOD/SCID/β2m^{null} (non-patent document 7), NOD-Rag1^{null} (non-patent document 8) and NOD-Rag1^{null}Prf1^{null} (non-patent document 9) (non-patent documents 10, 11).

The present inventors clarified that NOD/SCID/IL2rg KO mice maintain leukemia engraftment rates higher than do NOD/SCID/b2m KO mice, which are conventional immunodeficient mice becoming deficient not only in the acquired immune system, but also in the innate immune system. Furthermore, the present inventors showed that significantly higher engraftment rates are maintained by transplanting the graft in the neonatal stage than in the mature stage, which is used by many researchers for its technical convenience. Also, the present inventors found that recipient mice generated by transplanting LSCs derived from a human acute myelogenous leukemia (AML) patient to neonatal NOD/SCID/IL2rg^{null} mice well reproduced the pathologic condition of AML in each human patient, and are appropriate as a mouse model of AML. Furthermore, the present inventors found it possible to reproduce the leukemic state observed in patient bone marrow and propagate human AML cells (LSC and non-LSC), while maintaining the characters thereof, also by performing secondary and tertiary transplantation of LSCs obtained from a recipient mouse to another mouse. Furthermore, an analysis of the mice revealed that LSCs home in an osteoblast-rich region (niche) of bone marrow (BM) and engraft therein, where the LSCs have their cell cycle ceasing in the stationary phase and are hence protected against apoptosis induced by cell cycle-dependent chemotherapeutic agents (patent document 1, non-patent document 12). Therefore, it was thought that such LSCs having their cell cycle stationary do cause leukemia recurrence after chemotherapy.

By allowing cells in the stationary phase to initiate the progression of the cell cycle thereof, and concurrently applying a cell cycle-dependent chemotherapeutic agent, cell death such as due to apoptosis can be induced. While some cases are known where cytokines were allowed to act on a population of AML blast cells to reduce the colonizing potential thereof in vitro (non-patent documents 13 to 16), no investigation has been conducted to date to determine whether the effect was LSC-specific. Nor has it been thought at all that the progression of the cell cycle of LSCs as they are localized in the niche can be induced.

### PRIOR ART DOCUMENTS

### Patent Documents

[patent document 1] WO/2009/051238

### Non-Patent Documents

[non-patent document 1] Passegue, E., Jamieson, C. H., Ailles, L. E. & Weissman, I. L. Normal and leukemic hematopoiesis: are leukemias a stem cell disorder or a reacquisition of stem cell characteristics? Proc Natl Acad Sci USA 100 Suppl 1, 11842-11849 (2003).

[non-patent document 2] Hope, K. J., Jin, L. & Dick, J. E. Acute myeloid leukemia originates from a hierarchy of leukemic stem cell classes that differ in self-renewal capacity. Nat Immunol 5, 738-743 (2004).

[non-patent document 3] Jordan, C. T. & Guzman, M. L. Mechanisms controlling pathogenesis and survival of leukemic stem cells. Oncogene 23, 7178-7187 (2004).

[non-patent document 4] Cao, X. et al. Defective lymphoid development in mice lacking expression of the common cytokine receptor gamma chain. Immunity 2, 223-238 (1995).

[non-patent document 5] Ishikawa, F. et al. Development of functional human blood and immune systems in NOD/SCID/IL2 receptor {gamma} chain (null) mice. Blood 106, 1565-1573 (2005).

[non-patent document 6] Shultz, L. D. et al. Multiple defects in innate and adaptive immunologic function in NOD/LtSz-scid mice. J Immunol 154, 180-191 (1995).

[non-patent document 7] Christianson, S. W. et al. Enhanced human CD4+ T cell engraftment in beta2-microglobulin-deficient NOD-scid mice. J Immunol 158, 3578-3586 (1997).

[non-patent document 8] Shultz, L. D. et al. NOD/LtSz-Rag1null mice: an immunodeficient and radioresistant model for engraftment of human hematolymphoid cells, HIV infection, and adoptive transfer of NOD mouse diabetogenic T cells. Journal of Immunology 164, 2496-2507 (2000).

[non-patent document 9] Shultz, L. D. et al. NOD/LtSz-Rag1nullPfpnull mice: a new model system with increased levels of human peripheral leukocyte and hematopoietic stem-cell engraftment. Transplantation 76, 1036-1042 (2003).

[non-patent document 10] Huntly, B. J. et al. MOZ-TIF2, but not BCR-ABL, confers properties of leukemic stem cells to committed murine hematopoietic progenitors. Cancer Cell 6, 587-596 (2004).

[non-patent document 11] Shultz, L. D. et al. Human lymphoid and myeloid cell development in NOD/LtSz-scid IL2R gamma null mice engrafted with mobilized human hemopoietic stem cells. J Immunol 174, 6477-6489 (2005).

[non-patent document 12] Ishikawa, F. et al. Chemotherapy-resistant human AML stem cells home to and engraft within the bone-marrow endosteal region. Nat Biotechnol 25, 1315-1321 (2007) .

[non-patent document 13] Cannistra, S. A. et al. Granulocyte-macrophage colony-stimulating factor enhances the cytotoxic effects of cytosine arabinoside in acute myeloblastic leukemia and in the myeloid blast crisis phase of chronic myeloid leukemia. Leukemia 3, 328-34 (1989).

[non-patent document 14] Miyauchi, J. et al. Growth factors influence the sensitivity of leukemic stem cells to cytosine arabinoside in culture. Blood 73, 1272-1278 (1989).

[non-patent document 15] Andreeff, M. et al. Colony-stimulating factors (rhG-CSF, rhGM-CSF, rhIL-3, and BCGF) recruit myeloblastic and lymphoblastic leukemic cells and enhance the cytotoxic effects of cytosine-arabinoside. Haematol Blood Transfus 33, 747-762 (1990).

[non-patent document 16] te Boekhorst, P A. et al. Hematopoietic growth factor stimulation and cytarabine cytotoxicity in vitro: effects in untreated and relapsed or primary refractory acute myeloid leukemia cells. Leukemia 8, 1480-1486 (1994).

### SUMMARY OF THE INVENTION

### Problems to be Solved by the Invention

It is an object of the present invention to provide a method of killing leukemia stem cells to suppress and prevent leukemia recurrence, without relying on conventional chemotherapy alone, by initiating the progression of the cell cycle of leukemia stem cells in the stationary phase to make the leukemia stem cells sensitive to cell cycle-dependent chemotherapeutic agents.

### Means of Solving the Problems

As stated above, the present inventors elucidated that chemotherapy-refractory leukemia stem cells are localized in the niche in bone marrow (BM) (Nat Biotechnol 25, 1315-1321 (2007)), and that leukemia stem cells have their cell cycle stationary in the niche. Hence, mobilizing the cell cycle of leukemia stem cells in the niche is a key to overcoming recurrence. With this in mind, the present inventors searched for a drug capable of specifically initiating the progression of the cell cycle of leukemia stem cells that have their cell cycle ceasing in the stationary phase and cannot therefore be killed by cell cycle-dependent chemotherapeutic agents, even in the niche, using the above-described mouse model (NOD/SCID/IL2rg^{null}) of AML. As a result, the present inventors discovered that by administering granulocyte colony stimulation factor (G-CSF), initiation of the progression of the cell cycle of the LSCs can be induced in the niche as well in vivo. Furthermore, the present inventors demonstrated from a survival curve showing a significant extension in transplantation experiments that by administering in combination G-CSF and a cell cycle-dependent chemotherapeutic agent, apoptosis of the leukemia stem cells localized in the niche can be induced at extremely high efficiency, and, as a result, leukemia recurrence can be prevented, and have completed the present invention.

Accordingly, the present invention is as follows:
[1] An agent for inducing the progression of the cell cycle of leukemia stem cells, which comprises G-CSF.
[2] The agent according to [1], wherein the leukemia stem cells are in the stationary phase.
[3] The agent according to [2], wherein the leukemia stem cells are present in the niche in bone marrow.
[4] A medicament for killing leukemia stem cells, comprising a combination of G-CSF and a cell cycle-dependent antitumor agent.
[5] The medicament according to [4], wherein the cell cycle-dependent antitumor agent is administered after administration of G-CSF.
[6] A drug for suppressing leukemia, comprising a combination of G-CSF and a cell cycle-dependent antitumor agent.
[7] The drug according to [6], wherein the cell cycle-dependent antitumor agent is administered after administration of G-CSF.
[8] The drug according to [6], which is for suppressing recurrence of leukemia.
[9] A method of inducing the progression of the cell cycle of leukemia stem cells in a mammal, comprising administering G-CSF to the mammal.
[10] A method of killing leukemia stem cells in a mammal, comprising administering G-CSF and a cell cycle-dependent antitumor agent to the mammal.
[11] The method according to [10], wherein the cell cycle-dependent antitumor agent is administered after administration of G-CSF.
[12] A method of suppressing leukemia in a mammal, comprising administering G-CSF and a cell cycle-dependent antitumor agent to the mammal.
[13] The method according to [12], wherein the cell cycle-dependent antitumor agent is administered after administration of G-CSF.
[14] G-CSF for use in inducing the progression of the cell cycle of leukemia stem cells.
[15] A combination comprising G-CSF and a cell cycle-dependent antitumor agent for use in killing leukemia stem cells.
[16] The combination according to [15], wherein the cell cycle-dependent antitumor agent is administered after administration of G-CSF.
[17] A combination comprising G-CSF and a cell cycle-dependent antitumor agent for use in suppressing leukemia.
[18] The combination according to [17], wherein the cell cycle-dependent antitumor agent is administered after administration of G-CSF

### Effect of the Invention

By using the agent for inducing the progression of cell cycle of the present invention, it is possible to induce the progression of the cell cycle of leukemia stem cells that are localized in the niche in bone marrow (BM), and that have their cell cycle ceasing in the stationary phase. Because leukemia stem cells having their cell cycle progressing are more sensitive to cell cycle-dependent antitumor agents, it is possible to kill leukemia stem cells at high efficiency by administering in combination the agent for inducing the progression of cell cycle of the present invention and a cell cycle-dependent antitumor agent. Because leukemia stem cells are the major cause of leukemia recurrence, it is possible to suppress and prevent leukemia recurrence by killing leukemia stem cells.

### BRIEF DESCRIPTION OF THE DRAWINGS

FIG. 1 is a graphic representation showing that administration of G-CSF in vivo initiates the progression of the cell cycle of LSCs in the stationary phase. (A) Representative contour maps generated by a flow cytometric analysis of hCD34⁺CD38⁻ LSCs of the BM at baseline of recipients of primary transplantation of human AML in a constant state without administration of a drug such as G-CSF, after administration of cytarabine (Ara-C) in vivo, and after administration of G-CSF followed by administration of cytarabine in vivo. (B) With administration of G-CSF in vivo (open circles), the ratio of LSCs in the G0 phase of the cell cycle in the recipient BM decreased compared with the absence of administration of G-CSF (solid circles). Each horizontal bar indicates mean +SEM. Two-tailed t-test revealed p<0.005 in each case.

FIG. 2 shows that initiation of the progression of the cell cycle of AML cells that are present in the endosteal region is induced by G-CSF. (A) Shown are representative examples of bone sections from recipients of transplantation of human AML, derived from a recipient receiving administration of G-CSF in vivo or recipient not receiving the same, and immunohistochemically labeled with BrdU. This demonstrated that in relation to administration of G-CSF, AML in the endosteal region increases the uptake of BrdU (grey). (B) Immunofluorescence labeling with Ki67, a marker of the progression of the cell cycle, demonstrated that initiation of the progression of the cell cycle of AML cells in the endosteal region is induced by administration of G-CSF. Shown are images of CD34, Ki67, DAPI and a merged image thereof. Each scale bar indicates 20 μm (A) and 10 μm (B).

FIG. 3 shows that Ara-C-induced apoptosis is accentuated in the endosteal region of BM by pre-administration of G-CSF. (A) Representative histograms demonstrating that the expression of activated caspase-3 after chemotherapy is accentuated by pre-administration of G-CSF in human CD34⁺CD38⁻ LSCs and CD34⁺CD38⁺ AML non-stem cells derived from the BM of recipients of primary transplantation of human AML after administration of Ara-C alone in vivo, and after administration of G-CSF followed by administration of Ara-C in vivo. (B) In 7 recipients of transplantation of each type of LSCs, the survival of LSCs decreased with pre-administration of G-CSF followed by administration of Ara-C. Shown are percentages of BM LSCs that were negative for activated caspase-3 (i.e., resistant to anticancer agents) when Ara-C was administered alone (solid circles) or Ara-C was administered after administration of G-CSF (open circles). Two-tailed t-test revealed a significant difference in each case (p<0.05). (C) From HE staining and TUNEL staining of bone sections from recipients of transplantation of AML, it is evident that apoptosis is induced in the central region of BM with administration of Ara-C alone, but cells adjoining to the endosteum survive (*). In contrast, in the BM of recipients of administration of G-CSF followed by administration of Ara-C, cell death due to apoptosis was shown in the endosteal region (+), where treatment-refractory leukemia stem cells engraft, as well as in the central region. Each scale bar indicates 10 μm.

FIG. 4 shows that by combining pre-administration of G-CSF and administration of Ara-C, the frequency of LSCs is decreased and the survival of secondary recipients is improved. (A) Since leukemia recurrence/development has been proven to occur only from LSCs using the maximum likelihood method, the frequency of LSCs was estimated by Poisson statistics. In the analysis, positive transplantation was defined as hCD45⁺>1.0% in peripheral blood on week 18 after transplantation. *After administration, no sufficient number of hCD34+ cells for limited dilution transplantation could be isolated. **Because engraftment occurred in all recipients, frequency could not be estimated. ***Because engraftment did not occur in any recipient, frequency could not be estimated. P values were obtained by two-tailed t-test. The range indicates +/− SEM. (B) The survival at large of mice receiving viable hCD34+ AML cells derived from a recipient of transplantation of AML, receiving administration of Ara-C alone or administration of Ara-C in combination with G-CSF, was estimated by the Kaplan-Meier method. Comparisons within each administration level and among different administration levels, it was found that in secondary mouse recipients of transplantation of AML receiving administration of Ara-C in combination with G-CSF, the survival at large improved statistically significantly (by log-rank test, p<0.0001). Dose 2×10³ (solid line): Ara-C alone n=25, G-CSF+Ara-C n=21; dose 2×10⁴ (broken line): Ara-C alone n=22, G-CSF+Ara-C n=14; dose 2×10⁵ (broken line with dots): Ara-C alone n=15, G-CSF+Ara-C n=14.

### MODES FOR EMBODYING THE INVENTION

### (1) Use of G-CSF for Inducing the Progression of the Cell Cycle of Leukemia Stem Cells

The present invention provides an agent comprising G-CSF for inducing the progression of the cell cycle of leukemia stem cells.

G-CSF is a publicly known cytokine, whose amino acid sequence and the like are also publicly known. The G-CSF used in the present invention is normally derived from a mammal.

Being “derived from a mammal” means that the amino acid sequence of the G-CSF is a mammalian sequence. Mammals include, for example, laboratory animals such as mice, rats, hamsters, guinea pigs, and other rodents, and rabbits; domestic animals such as swines, cattle, goats, horses, sheep, and minks; companion animals such as dogs and cats; and primates such as humans, monkeys, cynomolgus monkeys, rhesus monkeys, marmosets, orangutans, and chimpanzees. The G-CSF used in the present invention is preferably derived from human.

Representative amino acid sequences of human G-CSF can include the amino acid sequence shown by SEQ ID NO:2 (full-length) and SEQ ID NO:3 (mature type resulting from cleavage of signal sequence). Herein, for proteins and peptides, the left end indicates the N-terminus (amino terminus) and the right end indicates the C-terminus (carboxyl terminus), according to the common practice of peptide designation.

Polypeptides that have a portion of the amino acid sequence of natural type G-CSF deleted, substituted, added and/or inserted, and that have granulocyte colony formation activity (G-CSF derivatives) are also included in the G-CSF used in the present invention. Such G-CSF derivatives are disclosed in, for example, Japanese Patent No. 2718426, Japanese Patent No. 2527365, Japanese Patent No. 2660178, Japanese Patent No. 2660179, JP-B-6-8317, Japanese Patent No. 2673099 and the like.

The G-CSF may be one isolated or purified from cells that produce the same or a culture supernatant thereof by a protein separation and purification technique known per se. The G-CSF may be a protein biochemically synthesized using a chemical synthesis or cell-free translation system, or may be a recombinant protein produced by a transformant introduced with a nucleic acid having the base sequence that encodes the aforementioned amino acid sequence.

It is preferable that the G-CSF used in the present invention have been isolated or purified. “Isolated or purified” means that an operation has been performed for removing components other than the desired component. The purity of the isolated or purified G-CSF (G-CSF relative to total polypeptide weight) is normally 50% by weight or more, preferably 70% or more, more preferably 90% or more, most preferably 95% or more (for example, substantially 100%).

The G-CSF used in the present invention may have been modified. The modification is exemplified by, but is not limited to, addition of lipid chain (aliphatic acylations (palmitoylation, myristoylation and the like), prenylations (farnesylation, geranylgeranylation and the like) and the like), phosphorylation (phosphorylation at serine residue, threonine residue, tyrosine residue and the like), acetylation, addition of sugar chain (N-glycosylation, O-glycosylation), addition of polyethylene glycol chain, and the like.

A leukemia stem cell refers to a cell that meets the following requirements:
1. Possesses the capability of causing leukemia in living organisms selectively and exclusively.
2. Capable of producing a leukemia non-stem cell fraction that cannot cause leukemia per se.
3. Capable of engrafting in living organisms.
4. Possesses a potential for self-replication.

Here, a potential for self-replication refers to the capability of division such that one of the two cells resulting from cell division becomes itself, i.e., a stem cell, and the other becomes a more differentiated progenitor cell. The concept of leukemia stem cells is already well established in the art and is widely accepted (D. Bonnet, J. E. Dick, Nat. Med. 3, 730 (1997) T. Lapidot et al., Nature 367, 645 (1994)).

Herein, leukemia stem cells encompass stem cells of all types of leukemia cells, preferably referring to stem cells of acute myelogenous leukemia cells.

The leukemia stem cells to which the agent of the present invention is applied are normally derived from a mammal. Mammals include, for example, laboratory animals such as mice, rats, hamsters, guinea pigs, and other rodents, and rabbits; domestic animals such as swines, cattle, goats, horses, sheep, and minks; companion animals such as dogs and cats; and primates such as humans, monkeys, cynomolgus monkeys, rhesus monkeys, marmosets, orangutans, and chimpanzees. The leukemia stem cells used in the present invention are preferably derived from a primate (for example, humans) or rodent (for example, mice).

Human leukemia cells normally have the hCD45⁺hCD33⁺ phenotype. Of human leukemia cells, leukemia stem cells normally have the hCD34⁺ phenotype. Of human leukemia stem cells, leukemia stem cells that selectively have the capability of causing leukemia, that have their cell cycle ceasing in the stationary phase, and that are resistant to chemotherapeutic agents, normally have the hCD38⁻ phenotype.

The cell cycle refers to the series of events that constitute cell division, including mitosis, cytokinesis and interphases, in eukaryotic organisms. In the cell, the first interphase (G1 phase) is followed by the DNA synthesis phase (S phase), in which DNA synthesis takes place. Upon completion of DNA synthesis, the second interphase (G2 phase) occurs in preparation for cell division. After the preparation is ready and genome replication is complete, the mitotic phase (M phase) occurs, in which cell division begins. The cell proliferates to two cells having the same genetic information, and returns to the first interphase (G1 phase). If growth stimulation on the cells continue, the cells proceed to the DNA synthesis phase (S phase), and the cell cycle is repeated. Without stimulation, the cells remain in the stationary phase (G0 phase).

“Induction of the progression of the cell cycle” refers to allowing cells in the stationary phase of the cell cycle to enter the cell cycle. Therefore, by inducing the progression of the cell cycle, cell division is initiated.

As shown in the Example below, the majority of leukemia stem cells are present in the bone marrow niche (the endosteal surface adjoining to a region where osteoblasts are abundantly present) and have their cell cycle ceasing. Furthermore, stem cells entering the cell cycle are killed by anticancer agents even if they have the phenotype CD34⁺CD38⁻, which is characteristic of stem cells. Therefore, it is critical in killing leukemia stem cells to cause the cells to leave the stationary phase in the cell cycle thereof and enter the G1, S, G2, M cycle. By applying G-CSF to leukemia stem cells, it is possible to allow the leukemia stem cells to enter the cell cycle, or to raise the turnover rate in the cell cycle, thereby to increase the sensitivity to cell cycle-dependent antitumor agents. Therefore, the agent of the present invention is useful as a medicament for increasing the sensitivity of leukemia stem cells to cell cycle-dependent antitumor agents. As stated below, by combining the agent of the present invention and a cell cycle-dependent antitumor agent, it is possible to efficiently kill leukemia stem cells.

The agent of the present invention can be administered as G-CSF as it is, or in an appropriate pharmaceutical composition, to human or non-human mammals (e.g., mice, rats, rabbits, sheep, swines, cattle, cats, dogs, monkeys and the like). The pharmaceutical composition used for the administration may comprise G-CSF and a pharmacologically acceptable carrier, diluent or excipient. Such a pharmaceutical composition is provided as a dosage form suitable for oral or parenteral administration.

Examples of compositions for parenteral administration include injections, suppositories and the like; the injection may include dosage forms such as intravenous injections, subcutaneous injections, intracutaneous injections, intramusclular injections, and drip injections. Such an injection can be prepared according to a publicly known method. Regarding how to prepare an injection, an injection can be prepared, for example, by dissolving, suspending or emulsifying the above-described G-CSF in a sterile aqueous liquid or oily liquid normally used for injections. The aqueous liquid for injections is exemplified by physiological saline, isotonic solutions containing glucose or other auxiliary agent, and may be used in combination with an appropriate solubilizer, for example, an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)] and the like. The oily liquid is exemplified by sesame oil, soybean oil and the like, and may be used in combination with a solubilizer such as benzyl benzoate or benzyl alcohol. The injectable preparation prepared is preferably filled in an appropriate ampoule. The suppository to be used for rectal administration may be prepared by mixing the above-described G-CSF in an ordinary suppository base.

Compositions for oral administration include solid or liquid dosage forms, specifically tablets (including sugar-coated tablets and film-coated tablets), pills, granules, powders, capsules (including soft capsules), syrups, emulsions, suspensions and the like. Such a composition is produced by a publicly known method, and may contain a carrier, diluent or excipient in common use in the field of medicament making. Useful carriers and excipients for tablets include, for example, lactose, starch, sucrose, magnesium stearate and the like.

Also, the agent of the present invention may be formulated with, for example, a buffering agent (for example, phosphate buffer solution, sodium acetate buffer solution), a soothing agent (for example, benzalkonium chloride, procaine hydrochloride and the like), a stabilizer (for example, human serum albumin, polyethylene glycol and the like), a preservative (for example, benzyl alcohol, phenol and the like), an antioxidant and the like. The prepared medicament can be filled in an appropriate ampoule.

The above-described pharmaceutical composition for parenteral or oral administration is conveniently prepared in a medication unit dosage form suitable for the dose of the active ingredient. Examples of such a medication unit dosage form include tablets, pills, capsules, injections (ampoules), aerosols and suppositories. Infusion pumps, transdermal patches and subcutaneously embedded agents are also included as methods of administration suitable for continuously obtaining a persistent drug effect. Regarding the content of G-CSF, it is preferable that normally 1 to 5000 mg, particularly 2 to 3000 mg for injections, or 5 to 3000 mg for other dosage forms, of the above-described G-CSF, per medication unit dosage form be contained.

The dose of the above-described preparation containing G-CSF varies depending on the recipient, symptoms, the route of administration and the like; for example, when using the same to induce the progression of the cell cycle of adult leukemia stem cells, it is convenient to administer G-CSF normally at about 0.01 to 50 mg/kg body weight, preferably at about 0.1 to 20 mg/kg body weight, more preferably at about 0.2 to 10 mg/kg body weight, based on a single dose, about 1 to 3 times a day, preferably once a day, by intravenous injection or drip infusion. In the case of other routes of parenteral administration (intramuscular administration, subcutaneous administration) and oral administration, amounts according to the above can be administered. In the case of a particularly severe symptom, the dose may be increased according to the symptom. The dosing frequency for G-CSF varies depending on the recipient, symptoms, the route of administration and the like, and is, for example, a frequency of once every 1 to 7 days, preferably a frequency of once every 1 to 3 days. The number of times of administration of G-CSF varies depending on the recipient, symptoms, the route of administration, the kind of antitumor agent and the like, and is normally about 1 to 15 times, preferably 2 to 10 times.

### (2) Combination of G-CSF and Cell Cycle-Dependent Antitumor Agent

The present invention further provides a medicament comprising a combination of G-CSF and a cell cycle-dependent antitumor agent.

A cell cycle-dependent antitumor agent means an antitumor agent that has a higher killing effect on cells having their cell cycle progressing than on cells having their cell cycle ceasing, because the active ingredient thereof targets a molecule or mechanism that is contributory to the progression of the cell cycle. The cell cycle-dependent antitumor agent is exemplified by, but is not limited to, drugs that are publicly known as chemotherapeutic agents for cancer, for example, alkylating agents (e.g., cyclophosphamide, iphosphamide and the like), metabolism antagonists (e.g., cytarabine, 5-fluorouracil, methotrexate and the like), anticancer antibiotics (e.g., Adriamycin and the like, mitomycin), plant-derived anticancer agents (e.g., vinblastine, vincristine, vindesine, taxol and the like), cisplatin, carboplatin, etoposide and the like. In particular, cytarabine, 5-fluorouracil and the like are preferred. Regarding “cell cycle-dependent antitumor agents”, detailed descriptions are given in, for example, a document, Brunton, L L. Parker, K L. and Lazo, J S., Goodman and Gillman's The Pharmacological Basis of Therapeutics. 11^{th}ed. McGraw Hill Publishing (2005), the Wikipedia's entry “Anticancer Agents” and the like.

The cell cycle-dependent antitumor agent used in the present invention is preferably one that is effective against leukemia (particularly acute myelogenous leukemia).

When using G-CSF and a cell cycle-dependent antitumor agent in combination, the dosing times of the G-CSF and cell cycle-dependent antitumor agent are not limited; the G-CSF and cell cycle-dependent antitumor agent may be administered to the recipient simultaneously or at a time lag. The doses of the G-CSF and cell cycle-dependent antitumor agent are not particularly limited, as far as the desired effect (killing of leukemia stem cells or suppression and prevention of leukemia) can be accomplished, and the doses can be chosen as appropriate according to the recipient, the route of administration, symptoms, combination and the like.

The mode of administration of G-CSF and a cell cycle-dependent antitumor agent is not particularly limited, as far as the G-CSF and cell cycle-dependent antitumor agent are combined at the time of administration. Examples of such modes of administration include (1) administration of a single preparation obtained by simultaneously preparing G-CSF and a cell cycle-dependent antitumor agent, (2) simultaneous administration via the same route of administration of two different preparations obtained by separately preparing G-CSF and a cell cycle-dependent antitumor agent, (3) administration at a time lag via the same route of administration of two different preparations obtained by separately preparing G-CSF and a cell cycle-dependent antitumor agent, (4) simultaneous administration via different routes of administration of two different preparations obtained by separately preparing G-CSF and a cell cycle-dependent antitumor agent, (5) administration at a time lag via different routes of administration of two different preparations obtained by separately preparing G-CSF and a cell cycle-dependent antitumor agent (for example, administration in the order of G-CSF→cell cycle-dependent antitumor agent, or administration in the reverse order) and the like.

The medicament of the present invention can be administered as a combination of G-CSF and a cell cycle-dependent antitumor agent as they are, or in an appropriate pharmaceutical composition, to human or non-human mammals (e.g., mice, rats, rabbits, sheep, swines, cattle, cats, dogs, monkeys and the like). The pharmaceutical composition used for the administration may comprise G-CSF and/or a cell cycle-dependent antitumor agent and a pharmacologically acceptable carrier, diluent or excipient. Such a pharmaceutical composition is provided as a dosage form suitable for oral or parenteral administration.

Examples of compositions for parenteral administration include injections, suppositories and the like; the injections may include dosage forms such as intravenous injections, subcutaneous injections, intracutaneous injections, intramuscular injections and drip infusion injections. Such an injection can be prepared according to a publicly known method. Regarding how to prepare an injection, an injection can be prepared by, for example, dissolving, suspending or emulsifying the above-described G-CSF and/or cell cycle-dependent antitumor agent in a sterile aqueous liquid or oily liquid normally used for injections. The aqueous liquid for injections is exemplified by physiological saline, isotonic solutions containing glucose or another auxiliary agent, and may be used in combination with an appropriate solubilizer, for example, an alcohol (e.g., ethanol), a polyalcohol (e.g., propylene glycol, polyethylene glycol), a nonionic surfactant [(e.g., Polysorbate 80, HCO-50 (polyoxyethylene (50 mol) adduct of hydrogenated castor oil)] and the like. The oily liquid is exemplified by sesame oil, soybean oil and the like, and may be used in combination with a solubilizer such as benzyl benzoate or benzyl alcohol. The injectable preparation prepared is preferably filled in an appropriate ampoule. The suppository to be used for rectal administration may be prepared by mixing the above-described G-CSF and/or cell cycle-dependent antitumor agent in an ordinary suppository base.

Compositions for oral administration include solid or liquid dosage forms, specifically tablets (including sugar-coated tablets and film-coated tablets), pills, granules, powders, capsules (including soft capsules), syrups, emulsions, suspensions and the like. Such a composition is produced by a publicly known method, and may contain a carrier, diluent or excipient in common use in the field of medicament making. Useful carriers and excipients for tablets include, for example, lactose, starch, sucrose, magnesium stearate and the like.

Also, the medicament of the present invention may be formulated with, for example, a buffering agent (for example, phosphate buffer solution, sodium acetate buffer solution), a soothing agent (for example, benzalkonium chloride, procaine hydrochloride and the like), a stabilizer (for example, human serum albumin, polyethylene glycol and the like), a preservative (for example, benzyl alcohol, phenol and the like), an antioxidant and the like. The prepared medicament can be filled in an appropriate ampoule.

The above-described pharmaceutical composition for parenteral or oral administration is conveniently prepared in a medication unit dosage form suitable for the dose of the active ingredient. Examples of such a medication unit dosage form include tablets, pills, capsules, injections (ampoules), aerosols and suppositories.

When G-CSF and a cell cycle-dependent antitumor agent are prepared as separate preparations, the G-CSF content in the medicament of the present invention is as described in the term (1).

The content of a cell cycle-dependent antitumor agent in the medicament of the present invention differs depending on the form of the preparation and the kind of antitumor agent, and is normally about 0.1 to 99.9% by weight, preferably about 1 to 99% by weight, more preferably about 10 to 90% by weight, relative to the entire preparation.

When G-CSF and a cell cycle-dependent antitumor agent are used as a single preparation prepared at the same time, the contents thereof may be ones according to the above. In this case, the blending ratio of G-CSF and the cell cycle-dependent antitumor agent can be chosen as appropriate according to the recipient, the route of administration, symptoms, the kind of cell cycle-dependent antitumor agent and the like.

The dose of G-CSF varies depending on the recipient, symptoms, the route of administration and the like; for example, when G-CSF is used to kill adult leukemia stem cells, it is convenient to administer G-CSF normally at about 0.01 to 50 mg/kg body weight, preferably at about 0.1 to 20 mg/kg body weight, more preferably at about 0.2 to 10 mg/kg body weight, based on a single dose, about 1 to 3 times a day, preferably once a day, by intravenous injection or drip infusion. In the case of other routes of parenteral administration and oral administration, amounts according to the above can be administered. In the case of a particularly severe symptom, the dose may be increased according to the symptom.

The dose of the cell cycle-dependent antitumor agent varies depending on the recipient, symptoms, the route of m administration, the kind of antitumor agent and the like; for example, when cytarabine is used to kill adult leukemia stem cells, it is convenient to administer cytarabine normally at about 0.01 to 2 g/kg body weight, preferably at about 0.05 to 1 g/kg body weight, more preferably at about 0.1 to 0.5 g/kg body weight, based on a single dose, about 1 to 3 times a day, preferably once a day, by intravenous injection or drip infusion. In the case of other routes of parenteral administration and oral administration, amounts according to the above can be administered. In the case of a particularly severe symptom, the dose may be increased according to the symptom.

The dosing frequency for G-CSF and/or the cell cycle-dependent antitumor agent varies depending on the recipient, symptoms, the route of administration, the kind of antitumor agent and the like, and is, for example, a frequency of once every 1 to 7 days, preferably a frequency of once every 1 to 3 days. The number of times of administration of G-CSF and/or the cell cycle-dependent antitumor agent varies depending on the recipient, symptoms, the route of administration, the kind of antitumor agent and the like, and is normally about 1 to 15 times, preferably 2 to 10 times.

When the above-described G-CSF and cell cycle-dependent antitumor agent are administered in combination as separately prepared preparations, the preparation containing G-CSF and the preparation containing the cell cycle-dependent antitumor agent may be administered at the same time; however, the preparation containing the cell cycle-dependent antitumor agent may be administered in advance, after which the preparation containing G-CSF may be administered, or the preparation containing G-CSF may be administered in advance, after which the preparation containing the cell cycle-dependent antitumor agent may be administered. When the same m are administered at a time lag, the time lag differs depending on the active ingredient administered, dosage form, and the method of administration; for example, when the preparation containing G-CSF is administered in advance, a method is available wherein the preparation containing the cell cycle-dependent antitumor agent is administered within 1 minute to 3 days after administration of the preparation containing G-CSF. When the preparation containing the cell cycle-dependent antitumor agent is administered in advance, a method is available wherein the preparation containing G-CSF is administered within 1 minute to 3 days after administration of the cell cycle-dependent antitumor agent.

Because leukemia stem cells are normally in the stationary phase outside the cell cycle or have a slow turnover rate of the cell cycle, as stated above, they exhibit resistance to cell cycle-dependent antitumor agents. By applying G-CSF to leukemia stem cells, it is possible to allow the leukemia stem cells to enter their cell cycle to thereby increase their sensitivity to cell cycle-dependent antitumor agents. By allowing a cell cycle-dependent antitumor agent to act on cells that have become more sensitive to cell cycle-dependent antitumor agents, it is possible, as a result, to kill leukemia stem cells at high efficiency. Therefore, by administering the medicament of the present invention to a mammal having leukemia stem cells, it is possible to kill the leukemia stem cells in the mammal.

Based on this theory, it is preferable that administration of a cell cycle-dependent antitumor agent take place simultaneously with administration of G-CSF or after a given period following administration of G-CSF, more preferably after a given period following administration of G-CSF. Hence, the dosing protocol for the medicament of the present invention preferably comprises a step for simultaneously administering G-CSF and a cell cycle-dependent antitumor agent, or a step for administering G-CSF and then administering a cell cycle-dependent antitumor agent, more preferably comprises a step for administering G-CSF and then administering a cell cycle-dependent antitumor agent. It is also preferable that initiation of the progression of the cell cycle of leukemia stem cells be confirmed after administration of G-CSF, and thereafter a cell cycle-dependent antitumor agent be administered.

Therefore, the dosing protocol for the medicament of the present invention preferably comprises the steps of:
(1) administering G-CSF and a cell cycle-dependent antitumor agent one time or a plurality of times,
(2) administering G-CSF one time or a plurality of times in a first stage, and administering a cell cycle-dependent antitumor agent one time or a plurality of times in a second stage,
(3) administering G-CSF one time or a plurality of times in a first stage, and administering G-CSF and a cell cycle-dependent antitumor agent one time or a plurality of times in a second stage,
(4) repeating the step (2) or (3) a plurality of times, and the like,
more preferably comprising any step selected from among (2) to (4) above.

In (2) and (3), the interval between the final administration in the first stage and the final administration in the second stage varies depending on the recipient, symptoms, the route of administration, the kind of antitumor agent and the like, and is normally within 1 minute to 3 days.

More specific examples of the steps in the aforementioned dosing protocol include, for example:
(1) administering G-CSF and a cell cycle-dependent antitumor agent at a frequency of once every 1 to 7 days, preferably at a frequency of once every 1 to 3 days, 1 to 15 times, preferably 2 to 10 times,
(2) administering G-CSF at a frequency of once every 1 to 7 days, preferably at a frequency of once every 1 to 3 days, 1 to 15 times, preferably 2 to 10 times, in a first stage, and administering a cell cycle-dependent antitumor agent at a frequency of once every 1 to 7 days, preferably at a frequency of once every 1 to 3 days, 1 to 15 times, preferably 2 to 10 times, in a second stage,
(3) administering G-CSF at a frequency of once every 1 to 7 days, preferably at a frequency of once every 1 to 3 days, 1 to 15 times, preferably 2 to 10 times, in a first stage, and administering G-CSF and a cell cycle-dependent antitumor agent at a frequency of once every 1 to 7 days, preferably at a frequency of once every 1 to 3 days, 1 to 15 times, preferably 2 to 10 times, in a second stage,
(4) repeating the step (2) or (3) a plurality of times, and the like.

Since leukemia stem cells are thought to a causal factor for leukemia recurrence, it is possible to suppress and prevent leukemia recurrence by using the medicament of the present invention. Hence, the medicament of the present invention is useful as a drug for suppressing leukemia (preferably a drug for suppressing recurrence of leukemia). Recurrence of leukemia means that complete or partial remission of a leukemia symptom by treatment is followed by re-growth of leukemia cells resulting in re-emergence or aggravation of the leukemia symptom. It is possible to suppress and prevent leukemia development (or recurrence) in a mammal by administering the medicament of the present invention to the mammal, wherein the mammal is at a risk of leukemia development (or recurrence).

### EXAMPLES

The present invention is hereinafter described in further detail by means of the following Examples, by which, however, the invention is not limited in any way.

### (Materials and Methods)

### Patient Samples

All experiments were performed with approval by the Institutional Review Board for Human Research at RIKEN's RCAI. AML patient-derived leukemia cells were collected with informed consent in writing. Samples were derived from AML patients having the French-American-British (FAB) classification system subtype M1 (not accompanied by maturation beyond premyelocytic leukemia; case 4), M2 (myeloblastic, accompanied by maturation; cases 3, 6, and 7), or M4 (myelomonocytic; cases 1 and 2). BMMNCs (bone marrow mononucleate cells) were isolated using density gradient centrifugation.

### Mice

NOD.Cg-Prkdc^{scid}Il2rg^{tmlWjl}/Sz (NOD/SCID/IL2rg^{null}) mice were developed at The Jackson Laboratory by backcrossing a complete null mutation (Shultz, L. D. et al. Multiple defects in innate and adaptive immunologic function in NOD/LtSz-scid mice. J Immunol 154, 180-191 (1995)) at the Il2rg locus onto the NOD.Cg-Prkdc^{scid} (NOD/SCID) strain. Mice were bred and maintained under defined flora with irradiated food and acidified water at the animal facility of RIKEN and at The Jackson Laboratory according to guidelines established by the Institutional Animal Committees at the respective institutions.

### Xenogeneic Transplantation

Newborn (within 2 days of birth) NOD/SCID/IL2rg^{null} recipient received 150 cGy of total body irradiation using a ¹³⁷Cs-source irradiator, followed by intravenous injection of AML cells within two hours. For primary transplantation, 10³ to 5×10⁴ sorted BM cells per recipient from a 7AAD⁻ lineage (hCD3/hCD4/hCD8) ⁻hCD34⁺hCD38⁻ AML patient were used, as described in F. Ishikawa et al., Nat. Biotechnol. 25, 1315 (2007). For secondary transplantation after administration of Ara-C (cytarabine) alone or after administration of G-CSF followed by administration of Ara-C, 2×10², 2×10³, 2×10⁴, or 2×10⁵ sorted 7AAD⁻hCD45⁺hCD34⁺ BM cells per recipient were used. For fluorescence-activated cell sorting, BMMNC cells from AML patients were labeled with fluorescent dye-conjugated mouse anti-hCD3, anti-hCD4, anti-hCD8, anti-hCD34 and anti-hCD38 monoclonal antibodies (BD Immunocytometry), and BMMNC cells from recipients were labeled with mouse anti-hCD45, anti-hCD34 and anti-hCD38 monoclonal antibodies (BD Immunocytometry); the cells were sorted using FACSAria (Beckton Dickinson, Calif.). Doublets were eliminated via analysis of FSC/SSC-height and FSC/SSC-width. After the sorting, the purities of hCD34⁺hCD38⁻ cells and hCD34⁺ cells exceeded 98%.

### Administration of G-CSF and Ara-C

For experiments involving administration of G-CSF alone, administration of Ara-C alone, and administration of G-CSF followed by administration of Ara-C, recipients of primary transplantation of human AML were used 16 to 24 weeks after transplantation. For each experiment for comparison of various dose groups, a pair of recipients was selected from among litter mates, with the same primary AML sample transplanted in the same amount on the same day so as to suppress variation among the litter mates and variation due to differences in transplantation level. Performed were administration of recombinant human G-CSF (Wako, Japan): 300 μg/kg s.c. qd×5 days; administration of Ara-C (Biogenesis, Poole, UK): 1 g/kg i.p. qd×2 days; administration of G-CSF+Ara-C: G-CSF 300 μg/kg s.c. qd×5 days, and concurrent administration of Ara-C 1 g/kg i.p. qd×2 days on days 4 and 5 of administration. The recipients were killed 16 hours after final injection. BrdU (1.5 mg/mouse; BD Biosciences, Calif.) was injected by i.p. to recipients under a cell cycle analysis by BrdU uptake immediately after the final injection (s.c. stands for subcutaneous administration, and i.p. for intraperitoneal administration).

### Flow Cytometry

For evaluation of human AML transplantation, blood was drawn from the orbital sinus of each recipient every 3 weeks starting at week 6 after transplantation. Myelocytes were recovered from two tibiae and one femur from each analyzed recipient; MNCs (mononucleocytes) were counted manually and using an automated blood cell analyzer (Celltac α, Nihon Kohden, Japan), and the absolute number of BMMNCs derived from each recipient was estimated. The absolute number of human CD34⁺ cells (derived from two tibiae and one femur) per mouse was determined by multiplying the thus-obtained total BMMNC count by 7AAD⁻hCD45⁺hCD34⁺ BM cells (%). BrdU uptake was measured using a BrdU flow kit (BD Pharmingen, Calif.). To quantify cells in the G0 phase of the cell cycle, the cells were stained with Hoechst 33342 and Pyronin Y, and then surface-stained using standard procedures. Quantitation of cells undergoing apoptosis was performed by staining activated caspase-3 in the cells using a rabbit anti-activated caspase-3 monoclonal antibody (BD Pharmingen, Calif.). Surface labeling was achieved using mouse anti-human CD45, anti-CD34 and anti-CD38 monoclonal antibodies (BD Immunocytometry). Analyses were performed using FACSAria and FACSCanto II (Becton Dickinson, Calif.).

### Histological Analysis and Immunofluorescent Imaging

Paraformaldehyde-fixed, decalcified, paraffin-embedded sections were prepared from femurs of the recipients primarily transplanted with AML. Mouse anti-human CD34 monoclonal antibody (Immunotech, France), rabbit anti-Ki67 polyclonal antibody (Spring Bioscience, Calif.) and mouse anti-BrdU monoclonal antibody (DAKO, Denmark) were used for antibody staining. Hematoxylin-eosin (HE) staining was performed according to a standard methodology. TUNEL staining was performed according to standard procedures using ApopTag peroxidase in situ apoptosis detection kit (Intergene, Purchase, N.Y.) by Biopathology Institute (Oita, Japan). Light microscopic observation was performed using Zeiss Axiovert 200 (Carl Zeiss, Germany). Laser-scanning confocal imaging was performed using Zeiss LSM Exciter and LSM 710 (Carl Zeiss, Germany).

### Statistical Analysis

Differences in the ratios/absolute numbers of cells (%), activated caspase-negative cells (%), and BM CD34⁺ cells in the cell cycle were analyzed using two-tailed t-test (GraphPad Prism, GraphPad, San Diego, Calif.). Differences in the number of viable cells were analyzed by log-rank (Mantel-Cox) test (GraphPad Prism, GraphPad, San Diego, Calif.). The frequency of LSCS was estimated by Poisson statistics using the maximum likelihood method and two-tailed t-test with L-Calc software (StemSoft Software, Vancouver, Canada).

### Example 1

First analyzed was the status of the progression of the cell cycle of LSCs and leukemia non-stem cells in the BM of NOD/SCID/IL2rg^{null} recipients of transplantation of LSCs obtained from the BM of seven AML patients. Although case-dependent variation existed, the ratios of cells in the G0 phase and those in the G1 phase were significantly higher in LSCs than in non-stem cells (hCD34⁺CD38⁺) in the BM of the recipients (Table 1).

TABLE 1
The cell cycle progresses vigorously in AML non-
stem cells, whereas the cell cycle has ceased in a larger
number of primary AML LSCs

| | | | within BM | within BM | |
|---|---|---|---|---|---|
| Case ID | n | | hCD34+CD38− | hCD34+CD38+ | p |
| 1 | 9 | % G0 | 64.8 +/− 5.1 | 20.3 +/− 3.9 | <0.0001 |
| | 8 | % G0/G1 | 84.6 +/− 1.4 | 55.8 +/− 7.5 | <0.01 |
| | 8 | % S | 8.4 +/− 1.6 | 26.9 +/− 6.9 | <0.05 |
| | 8 | % G2/M | 2.5 +/− 0.7 | 12.6 +/− 3.2 | <0.01 |
| 2 | 9 | % G0 | 31.5 +/− 2.0 | 8.3 +/− 1.1 | <0.0001 |
| | 7 | % G0/G1 | 86.1 +/− 4.0 | 52.7 +/− 5.1 | <0.0005 |
| | 7 | % S | 8.6 +/− 3.1 | 23.8 +/− 3.1 | <0.005 |
| | 7 | % G2/M | 2.2 +/− 0.6 | 19.9 +/− 5.2 | <0.005 |
| 3 | 11 | % G0 | 55.8 +/− 4.4 | 20.4 +/− 4.0 | <0.0001 |
| | 13 | % G0/G1 | 79.1 +/− 2.9 | 55.7 +/− 2.7 | <0.0001 |
| | 13 | % S | 12.3 +/− 2.3 | 23.0 +/− 2.1 | <0.005 |
| | 13 | % G2/M | 3.3 +/− 0.6 | 17.3 +/− 2.2 | <0.0001 |
| 4 | 6 | % G0 | 44.2 +/− 6.0 | 14.1 +/− 0.9 | <0.001 |
| | 7 | % G0/G1 | 82.1 +/− 5.6 | 57.7 +/− 3.8 | <0.005 |
| | 7 | % S | 9.5 +/− 3.1 | 19.2 +/− 1.3 | <0.05 |
| | 7 | % G2/M | 2.9 +/− 1.3 | 17.4 +/− 3.2 | <0.01 |
| 5 | 4 | % G0 | 50.0 +/− 6.4 | 17.9 +/− 9.5 | <0.05 |
| | 5 | % G0/G1 | 72.0 +/− 5.2 | 46.4 +/− 6.5 | <0.005 |
| | 5 | % S | 12.4 +/− 1.7 | 25.4 +/− 5.1 | <0.05 |
| | 5 | % G2/M | 5.5 +/− 1.3 | 24.9 +/− 8.0 | <0.05 |
| 6 | 6 | % G0 | 23.8 +/− 3.6 | 9.1 +/− 1.0 | <0.005 |
| | 5 | % G0/G1 | 79.0 +/− 3.2 | 31.3 +/− 3.5 | <0.0001 |
| | 5 | % S | 14.6 +/− 2.8 | 33.7 +/− 1.4 | <0.0005 |
| | 5 | % G2/M | 2.3 +/− 0.7 | 23.8 +/− 4.7 | <0.005 |
| 7 | 3 | % G0 | 67.8 +/− 5.6 | 20.3 +/− 5.2 | <0.005 |
| | 4 | % G0/G1 | 81.2 +/− 3.8 | 43.6 +/− 9.7 | <0.05 |
| | 4 | % S | 7.8 +/− 0.6 | 31.0 +/− 9.2 | <0.05 |
| | 4 | % G2/M | 2.7 +/− 0.8 | 20.8 +/− 2.9 | <0.005 |

In the BMMNCs obtained from the recipients of AML transplantation, CD34⁺CD38⁻ LSCs and CD34⁺CD38⁺ AML non-stem cells were compared. The results are shown as mean value +/− SEM; differences were tested by two-tailed t-test.

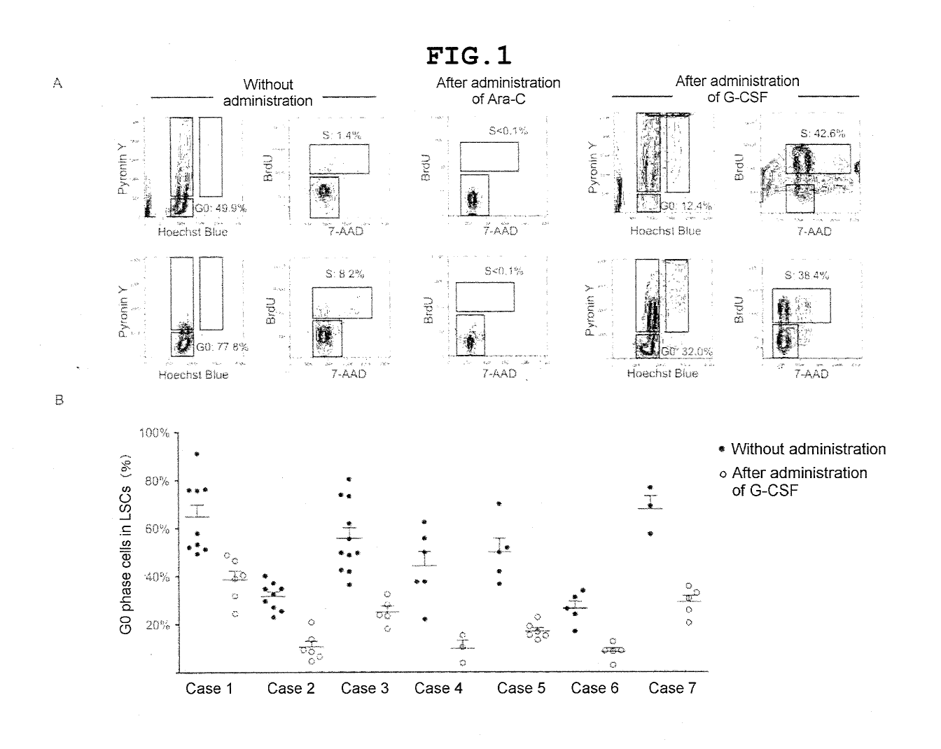

Next, the relationship between the status of the progression of the cell cycle of LSCs and the cytotoxic effect of the chemotherapeutic agent cytarabine (Ara-C) was analyzed. When Ara-C was intraperitoneally administered to NOD/SCID/IL2rg^{null} recipients of primary transplantation of AML, CD34⁺CD38⁻ AML cells in the S phase of the cell cycle were selectively eliminated, whereas CD34⁺CD38⁻ AML cells in the G0/G1 phase were relatively highly resistant, and were concentrated (% S=0.1+/−0.1 and % G0/G1=91.7+/−2.3 post-Ara-C, n=15; two-tailed t-test compared with non-administration recipients revealed p<0.0005; a representative data set of flow cytometry is shown in FIG. 1A).

Since CD34⁺CD38⁻ AML cells having their cell cycle progressing is selectively eliminated by Ara-C, it was hypothesized that the sensitivities thereof to chemotherapeutic agents are increased by inducing LSCs in the stationary phase to enter the cell cycle. To verify this hypothesis, the effect of administration of granulocyte colony stimulation factor (G-CSF) was analyzed in recipients of transplantation of AML in vivo. While it is well described that the cell cycle is induced by G-CSF in human and mouse HSCs, the effect of G-CSF on LSCs has not been proven accurately. Therefore, first, an analysis was performed to determine whether the status of the progression of the cell cycle of CD34⁺CD38⁻ LSCs changes with administration of G-CSF in recipients of primary transplantation of AML in vivo. A representative data set of flow cytometry is shown in FIG. 1A. In all cases examined, of the LCSs of recipients receiving transplantation of AML given administration of G-CSF, cells in the G0 phase fraction decreased significantly, and concurrently LSCs in the S phase and G2/M phase increased.

### Example 2

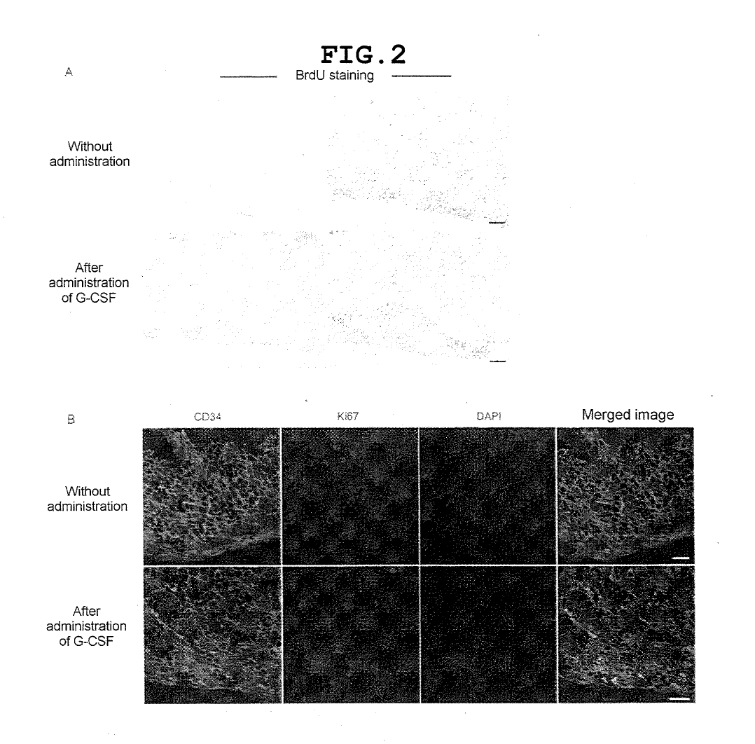

The present inventors previously demonstrated that CD34⁺CD38⁻ LSCs are present selectively in the endosteal region of BM, whereas CD38⁺ leukemia non-stem cells are detected mainly in the central region of BM. It is important that LSCs adjoining to the BM endosteum exhibit relatively high resistance to chemotherapy in vivo (F. Ishikawa et al., Nat. Biotechnol. 25, 1315 (2007)). Therefore, to directly evaluate the status of the progression of the cell cycle of LSCs in the BM endosteal niche, histological analysis was performed on recipients of primary transplantation of human AML (FIG. 2). In a constant state without administration of a drug such as G-CSF, leukemia cells in the central region of BM were strongly BrdU-positive; these cells exhibited high proliferation capability, whereas AML cells adjoining to the endosteum were found to be negative for BrdU staining; it was shown that these cells did not have a vigorous progression of the cell cycle (upper panel in FIG. 2A). In contrast, after administration of G-CSF, as is seen by the increase in BrdU uptake, AML cells in the endosteal region initiated the progression of their cell cycle (lower panel in FIG. 2A). Likewise, immunofluorescence staining with Ki67, which binds to a constituent of the nucleolus in the G1-S-G2 phase revealed that in a constant state without administration of a drug such as G-CSF, the majority of leukemia cells adjoining to the endosteum do not have a vigorous progression of their cell cycle (upper panel in FIG. 2B). Consistent with the finding of BrdU uptake assay in vivo, the expression of Ki67 was induced in the AML cells in the BM center after 5 days of administration of G-CSF, as well as in the AML cells in the endosteal region (lower panel in FIG. 2B). These flow cytometric findings and histological findings showed that G-CSF induces initiation of the progression of the cell cycle in LSCs in the stationary phase that are present in the endosteal niche.

### Example 3

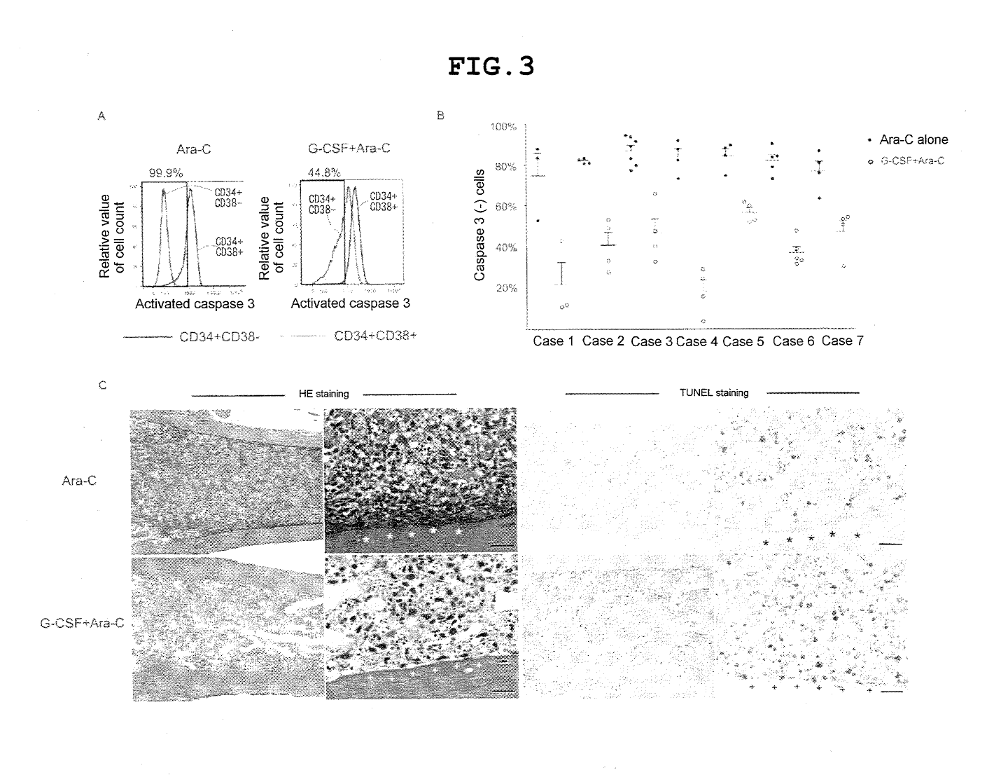

Next, to demonstrate that the sensitivity of LSCs to chemotherapy increases with initiation of the progression of the cell cycle, an in vivo model for evaluating the effects of administration of Ara-C alone and administration of Ara-C following pre-administration of G-CSF on LSCs in recipients of primary transplantation of AML was developed. After administration of Ara-C alone or after administration of Ara-C following pre-administration of G-CSF, the BM of the recipients was evaluated in terms of 1) a flow cytometry fraction of activated caspase-3 positive LSCs undergoing apoptosis, 2) histological localization of cells undergoing apoptosis in the recipient BM as determined by TUNEL staining, 3) percentage and absolute number of remaining viable hCD34⁺ AML cells, and 4) frequency and AML-causing potential of remaining LSCs in alternative measurements of the likelihood of AML recurrence via limited dilution and sequential transplantation of sorted hCD34⁺ cells. As shown in FIG. 3A, with administration of Ara-C alone in vivo, CD34⁺CD38⁺ AML non-stem cells underwent apoptosis, whereas the majority of CD34⁺CD38⁻ LSCs did not. In contrast, with administration of G-CSF+Ara-C, the frequency of activated caspase-3-negative LSCs decreased; it was shown that cell death due to apoptosis increased (FIG. 3B). Although variation in this effect was noted among the AML samples from the seven cases reflecting biological heterogeneity among the cases (i.e., individual differences), a statistically significant difference existed in that “leukemia stem cells were unlikely to get killed when the anticancer agent was administered alone, but a larger number of leukemia stem cells were killed by mobilizing the cell cycle” in all cases. A concurrently performed direct analysis of BM showed that with administration of Ara-C alone, the recipients had TUNEL-negative AML cells remaining in the endosteum (FIG. 3C). However, with administration of G-CSF+Ara-C, as demonstrated by both the reduction in cellularity revealed by HE staining and TUNEL staining positivity in the remaining cells, more efficient cell death was observed in the endosteum (and central region) in the recipients (FIG. 3C).

### Example 4

To evaluate the frequency and function of LSCs remaining after each dosing, limited dilution and secondary transplantation of living hCD34⁺ BM cells, including leukemia stem cells sorted from recipients given administration of Ara-C alone or G-CSF+Ara-C, were performed. The absolute number of hCD34⁺ cells was obtained from the number of mononucleocytes in two tibiae and one femur derived from each recipient, and viable hCD34⁺ cell (%) was obtained by flow cytometry. This demonstrated that in the BM of recipients given administration of G-CSF+Ara-C, the number of viable hCD34⁺ cells decreased significantly (Table 2).

TABLE 2
The frequency and number of hCD34⁺ cells,
including LSCs, decrease in vivo with administration of
Ara-C in combination with pre-administration of G-CSF

| Case ID | | Ara-C | G-CSF + Ara-C | p |
|---|---|---|---|---|
| 1 | % CD45+CD34+ | 29.7 +/− 1.6 | 15.5 +/− 3.8 | <0.05 |
| | #CD45+CD34+/ | 1.4 +/− 0.3 | 0.2 +/− 0.1 | <0.01 |
| | mouse (×10⁶) | | | |
| | n | 5 | 4 | |
| 2 | % CD45+CD34+ | 84.9 +/− 2.5 | 47.0 +/− 12.5 | <0.05 |
| | #CD45+CD34+/ | 4.9 +/− 0.7 | 1.5 +/− 0.5 | <0.01 |
| | mouse (×10⁶) | | | |
| | n | 4 | 4 | |
| 3 | % CD45+CD34+ | 80.2 +/− 2.3 | 54.4 +/− 4.4 | <0.005 |
| | #CD45+CD34+/ | 2.3 +/− 0.1 | 1.5 +/− 0.2 | <0.05 |
| | mouse (×10⁶) | | | |
| | n | 8 | 5 | |
| 4 | % CD45+CD34+ | 68.0 +/− 2.8 | 17.5 +/− 4.8 | <0.005 |
| | #CD45+CD34+/ | 3.6 +/− 0.8 | 0.5 +/− 0.1 | <0.05 |
| | mouse (×10⁶) | | | |
| | n | 4 | 4 | |
| 5 | % CD45+CD34+ | 61.5 +/− 7.5 | 20.7 +/− 0.6 | <0.005 |
| | #CD45+CD34+/ | 2.1 +/− 0.2 | 0.4 +/− 0.1 | <0.005 |
| | mouse (×10⁶) | | | |
| | n | 3 | 4 | |
| 6 | % CD45+CD34+ | 49.0 +/− 9.2 | 33.5 +/− 4.2 | <0.05 |
| | #CD45+CD34+/ | 5.3 +/− 1.4 | 1.3 +/− 0.2 | <0.0005 |
| | mouse (×10⁶) | | | |
| | n | 4 | 7 | |
| 7 | % CD45+CD34+ | 16.5 +/− 1.7 | 6.5 +/− 2.0 | <0.05 |
| | #CD45+CD34+/ | 0.7 +/− 0.1 | 0.2 +/− 0.1 | <0.005 |
| | mouse (×10⁶) | | | |
| | n | 5 | 5 | |

The flow cytometric analysis of the BM obtained from the two tibiae and one femur derived from recipients of transplantation demonstrated that in the recipients of transplantation of AML with pre-administration of G-CSF followed by administration of Ara-C, both the ratio and absolute number of viable hCD45⁺CD34⁺ cells decreased. The results are shown as mean value +/− SEM; differences were examined by two-tailed t-test.

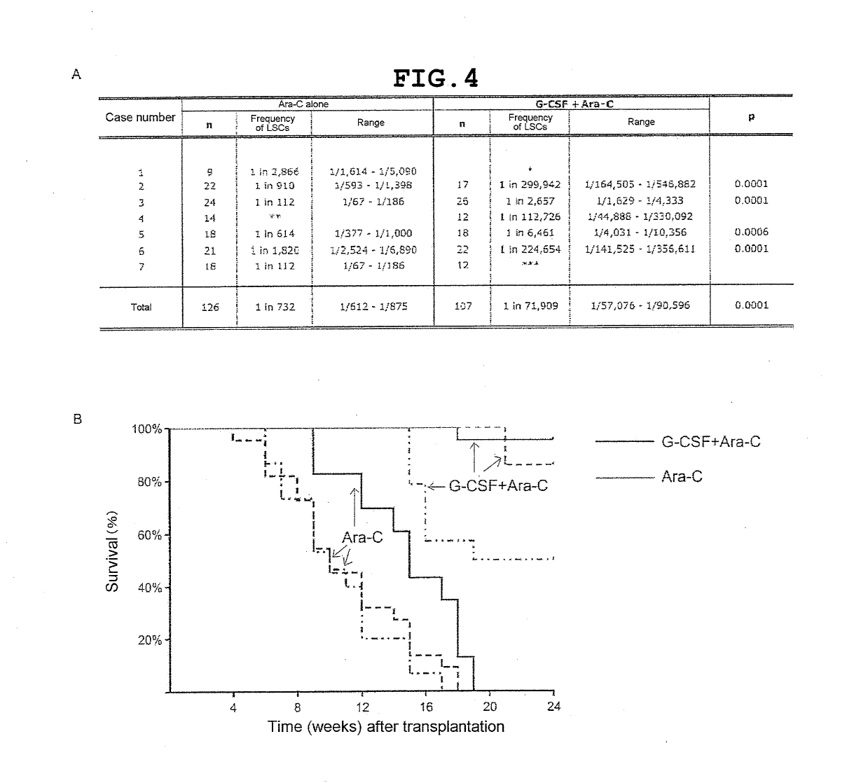

To definitely determine the function and frequency of LSCs remaining after each administration, viable hCD34⁺ BM cells were sorted, and re-transplanted to secondary recipients at doses of 2×10², 2×10³, 2×10⁴ and 2×10⁵ cells per recipient (FIG. 4). The frequency of LSCs was estimated by Poisson statistics, which is a standard methodology used to estimate the frequency of HSCs by limited dilution (referring to a method wherein a series of different numbers of stem cells are transplanted). As shown in FIG. 4, the estimated frequency of LSCs that are causal cells for recurrence was found to be significantly lower in the BM CD34⁺ population of the recipients given administration of G-CSF+Ara-C. Furthermore, 24 weeks after transplantation, in the secondary recipients of hCD34⁺ cells derived from a mouse receiving administration of G-CSF+Ara-C, a statistically significant improvement in survival was revealed at all doses (FIG. 4B). None of the secondary mouse recipients with administration of Ara-C alone survived beyond 19 weeks after transplantation, whereas 79.6% (39/49) of the secondary mouse recipients receiving administration of G-CSF+Ara-C survived beyond 24 weeks after transplantation; therefore, as leukemia stem cells were mostly killed, and recurrence was significantly suppressed, by administration of G-CSF+Ara-C, an efficacy of the present invention was demonstrated.

### INDUSTRIAL APPLICABILITY

According to the present invention, it is possible to provide an agent for suppressing recurrence of leukemia that dramatically improves the therapeutic efficiency for leukemia, which is extremely intractable so that the mean survival period, a patient prognostic factor, expected with conventional standard therapies, is about 1 year.

This application is based on a patent application No. 2009-052723 filed Mar. 5, 2009 in Japan, the contents of which are incorporated in full herein.

1. (canceled)

2. The method according to claim 9, wherein the leukemia stem cells are in the stationary phase.

3. The method according to claim 9, wherein the leukemia stem cells are present in the niche in bone marrow.

4.-7. (canceled)

8. The method according to claim 12, which is for suppressing recurrence of leukemia.

9. A method of inducing the progression of the cell cycle of leukemia stem cells in a mammal, comprising administering G-CSF to the mammal.

10. A method of killing leukemia stem cells in a mammal, comprising administering G-CSF and a cell cycle-dependent antitumor agent to the mammal.

11. The method according to claim 10, wherein the cell cycle-dependent antitumor agent is administered after administration of G-CSF.

12. A method of suppressing leukemia in a mammal, comprising administering G-CSF and a cell cycle-dependent antitumor agent to the mammal.

13. The method according to claim 12, wherein the cell cycle-dependent antitumor agent is administered after administration of G-CSF.

14.-18. (canceled)
